(19) United States
(12) Patent Application Publication
(10) Pub. No.: US 2003/0148287 A1
(43) Pub. Date: Aug. 7, 2003

(54) Libraries and kits for detecting transcription factor activity

(76) Inventors: Xianqiang Li, Palo Alto, CA (US); Xin Jiang, Saratoga, CA (US)

Correspondence Address: WILSON SONSINI GOODRICH & ROSATI, 650 PAGE MILL ROAD, PALO ALTO, CA, 943041050

(21) Appl. No.: 10/057,828

(22) Filed: Jan. 24, 2002

(51) Int. Cl. C12Q 1/68; G01N 33/53; C07H 21/04

(52) U.S. Cl. 435/6; 536/23.2

(57) ABSTRACT

A library of nucleic acid constructs is provided in which each construct comprises a cis element sequence comprising one or more copies of a cis element to which a transcription factor is capable of binding the cis element sequence varying within the library of constructs, a promoter sequence 3′ relative to the cis element sequence, a reporter sequence 3′ relative to the promoter sequence that comprises a variable sequence that varies within the library and wherein a same cis element sequence is employed with a given reporter sequence within the library of constructs.

### FIELD OF THE INVENTION

The present invention relates to methods for detecting transcription factor activity within a cell. More specifically, the invention relates to methods for detecting transcription factor activity within a cell sample for multiple transcription factors in parallel, as well as compositions, kits, and methods arising therefrom.

### DESCRIPTION OF RELATED ART

All living organisms use nucleic acids (DNA and RNA) to encode the genes that make up the genome for that organism. Each gene encodes a protein that may be produced by the organism through expression of the gene.

It is important to note that the mere presence of a gene in a cell does not communicate the functionality of that gene to the cell. Rather, it is only when the gene is expressed and a protein is produced that the functionality of the gene encoding the protein is conveyed.

Systems that regulate gene expression respond to a wide variety of developmental and environmental stimuli, thus allowing each cell type to express a unique and characteristic subset of its genes, and to adjust the dosage of particular gene products as needed. The importance of dosage control is underscored by the fact that targeted disruption of key regulatory molecules in mice often results in drastic phenotypic abnormalities [Johnson, R. S., et al., Cell, 71:577-586 (1992)], just as inherited or acquired defects in the function of genetic regulatory mechanisms contribute broadly to human disease.

The importance of controlled gene expression in human disease and the information available to date relating to the mechanisms of gene regulation have fueled efforts aimed at discovering ways of overriding endogenous regulatory controls or of creating new signaling circuitry in cells [Belshaw, P. J., et al., Proc. Natl. Acad. Sci. USA, 93:4604-4607 (1996); Ho, S. H., et al., Nature (London), 382:822-826 (1996); Rivera, V. M., et al., Nat. Med., 2:1028-1032; Spencer, D. M., et al., Science, 262:1019-1024 (1993)].

Critical to this research are effective tools for monitoring gene expression. It is therefore of interest to be able to rapidly and accurately determine the relative expression of different genes in different cells, tissues and organisms, over time, and under various conditions, treatments and regimes. As will be described herein in greater detail, there are a great many applications that arise from being able to effectively monitor which genes are being expressed by a given cell at a given time.

Standard molecular biology techniques have been used to analyze the expression of genes in a cell by measuring mRNA or protein expression.. These techniques include RT-PCR, Northern blot analysis, or other types of mRNA probe analysis such as in situ hybridization. Each of these methods allows one to analyze the transcription of only known genes and/or small numbers of genes at a time. Nucl. Acids Res. 19, 7097-7104 (1991); Nucl. Acids Res. 18, 4833-4842 (1990); Nucl. Acids Res. 18, 2789-2792 (1989); European J. Neuroscience 2, 1063-1073 (1990); Analytical Biochem. 187, 364-373 (1990); Genet. Annal Techn. Appl. 7, 64-70 (1990); GATA 8(4), 129-133 (1991); Proc. Natl. Acad. Sci. USA 85, 1696-1700 (1988); Nucl. Acids Res. 19, 1954 (1991); Proc. Natl. Acad. Sci. USA 88, 1943-1947 (1991); Nucl. Acids Res. 19, 6123-6127 (1991); Proc. Natl. Acad. Sci. USA 85, 5738-5742 (1988); Nucl. Acids Res. 16, 10937 (1988).

Gene expression has also been monitored by measuring levels of mRNA. Since proteins are transcribed from mRNA, it is possible to detect transcription by measuring the amount of mRNA present. One common method, called “hybridization subtraction”, allows one to look for changes in gene expression by detecting changes in mRNA expression. Nucl. Acids Res. 19, 7097-7104 (1991); Nucl. Acids Res. 18, 4833-4842 (1990); Nucl. Acids Res. 18, 2789-2792 (1989); European J. Neuroscience 2, 1063-1073 (1990); Analytical Biochem. 187, 364-373 (1990); Genet. Annal Techn. Appl. 7, 64-70 (1990); GATA 8(4), 129-133 (1991); Proc. Natl. Acad. Sci. USA 85, 1696-1700 (1988); Nucl. Acids Res. 19, 1954 (1991); Proc. Natl. Acad. Sci. USA 88, 1943-1947 (1991); Nucl. Acids Res. 19, 6123-6127 (1991); Proc. Natl. Acad. Sci. USA 85, 5738-5742 (1988); Nucl. Acids Res. 16, 10937 (1988).

Gene expression has also been monitored by measuring levels of gene product, (i.e., the expressed protein), in a cell, tissue, organ system, or even organism. Measurement of gene expression by measuring the protein gene product may be performed using antibodies known to bind to a particular protein to be detected. A difficulty arises in needing to generate antibodies to each protein to be detected. Measurement of gene expression via protein detection may also be performed using 2-dimensional gel electrophoresis, wherein proteins can be, in principle, identified and quantified as individual bands, and ultimately reduced to a discrete signal. In order to positively analyze each band, each band must be excised from the membrane and subjected to protein sequence analysis using Edman degradation. Unfortunately, it tends to be difficult to isolate a sufficient amount of protein to obtain a reliable sequence. In addition, many of the bands contain more than one discrete protein.

A further difficulty associated with quantifying gene expression by measuring an amount of protein gene product in a cell is that protein expression is an indirect measure of gene expression. It is impossible to know from a protein present in a cell when that protein was expressed by the cell. As a result, it is hard to determine whether protein expression changes over time due to cells being exposed to different stimuli.

Gene expression has also been monitored by measuring the amount of particular activated transcription factors present in a cell. Transcription in a cell is controlled by proteins, referred to herein as “activated transcription factors” that bind to DNA at sites outside the core promoter for the gene and activate transcription. Since activated transcription factors activate transcription, detection of their presence is useful for measuring gene expression. Transcriptional activators are found in prokaryotes, viruses, and eukaryotes, including fungi, plants, and animals, including mammals, providing a wide range of therapeutic targets.

The regulatory mechanisms controlling the transcription of protein-coding genes by RNA polymerase II have been extensively studied. RNA polymerase II and its host of associated proteins are recruited to the core promoter through non-covalent contacts with sequence-specific DNA binding proteins [Tjian, R. and Maniatis, T., Cell, 77:5-8 (1994); Stringer, K. F., Nature (London), 345:783-786 (1990)]. An especially prevalent and important subset of such proteins, known as transcription factors, typically bind DNA at sites outside the core promoter and activate transcription through space contacts with components of the transcriptional machinery, including chromatin remodeling proteins [Tjian, R. and Maniatis, T., Cell, 77:5-8 (1994); Stringer, K. F., Nature (London), 345:783-786 (1990); Bannister, A. J. and Kouzarides, T., Nature, 384:641-643 (1996); Mizzen, C. A., et al., Cell, 87:1261-1270 (1996)]. The DNA-binding and activation functions of transcription factors generally reside on separate domains whose operation is portable to heterologous fusion proteins [Sadowski, I., et al., Nature, 335:563-564 (1988)]. Though it is believed that activation domains are physically associated with a DNA-binding domain to attain proper function, the linkage between the two need not be covalent [Belshaw, P. J., et al., Proc. Natl. Acad. Sci. USA, 93:4604-4607 (1996); Ho, S. H., et al., Nature (London), 382:822-826 (1996)]. In many instances, the activation domain does not appear to contact the transcriptional machinery directly, but rather through the intermediacy of adapter proteins known as coactivators [Silverman, N., et al., Proc. Natl. Acad. Sci. USA, 91:11005-11008 ((1994); Arany, Z., et al., Nature (London), 374:81-84 (1995)].

One of the difficulties associated with measuring gene expression by measuring transcription factors is that one must measure the subset of transcription factors that are “activated.” Certain post-transcriptional modifications occur that render transcription factors “active” in the sense that they are capable of binding to DNA. It is thus necessary to distinguish between activated and non-activated transcription factors so that the “activated transcription factors” can be selectively measured.

Several different methods have been developed for detecting activated transcription factors. One method involves using antibodies selective for activated transcription factors over inactive forms of the transcription factor. This method is impractical for detecting multiple different activated transcription factors due to difficulties associated with developing numerous different antibodies having the requisite bind specificities.

Another method for detecting activated transcription factors involves measuring DNA—transcription factor complexes through a gel shift assay. [Ausebel, F. M. et al eds (1993) Current Protocols in Molecular Biology Vol.2 Greene Publishing Associates, Inc. and John Wiley and Sons, Inc., New York]. According to this method, a sample containing an activated transcription factor is contacted with a DNA probe that comprises a recognition sequence for the transcription factor. A complex between the activated transcription factor and the DNA probe is formed. The DNA-protein complex is detected by a gel-shift assay. Since individual gel shift assays must be performed for each activated transcription factor—DNA complex, this method is currently impractical for measuring multiple different activated transcription factors at the same time.

U.S. Pat. Nos. 6,066,452 and 5,861,246 describe methods for determining DNA binding sites for DNA-binding proteins. The DNA binding sites may then be used as probes to isolate DNA-binding proteins. Similarly, PCT Publication No. WO 00/04196 describes methods for identifying cis acting nucleic acid elements as well as methods for isolating nucleic acid binding factors.

Recently, application Ser. Nos. 09/877,738, 09/877,243, 09/877,403, 09/877,705, and 09/947,274 were filed by Applicant directed to methods for detecting activated transcription factors by detecting DNA probe—transcription factor complexes.

### SUMMARY OF THE INVENTION

The present invention relates to methods for detecting transcription factor activity in a cell sample for multiple different transcription factors. Applications of these methods are also provided. Compositions, libraries, and kits that may be used to perform these methods and applications are also provided.

In one embodiment, a library of nucleic acid constructs is provided, each construct comprising: a cis element sequence comprising one or more copies of a cis element to which a transcription factor is capable of binding, the cis element sequence varying within the library of constructs; a promoter sequence 3′ relative to the cis element sequence; and a reporter sequence 3′ relative to the promoter sequence that comprises a variable sequence that varies within the library; wherein a same cis element sequence is employed with a given reporter sequence within the library of constructs.

In another embodiment, a library of expression vectors is provided comprising: a library of constructs, each construct comprising a cis element sequence comprising one or more copies of a cis element to which a transcription factor is capable of binding, the cis element sequence varying within the library of constructs; a promoter sequence 3′ relative to the cis element sequence; and a reporter sequence 3′ relative to the promoter sequence that comprises a variable sequence that varies within the library of constructs; wherein a same cis element sequence is employed with a given reporter sequence within the library of constructs. According to this embodiment, the expression vectors are optionally mammalian expression vectors.

In another embodiment, a library of cells transduced or transfected with a library of constructs is provided, each construct comprising: a cis element sequence comprising one or more copies of a cis element to which a transcription factor is capable of binding, the cis element sequence varying within the library of constructs; a promoter sequence 3′ relative to the cis element sequence; and a reporter sequence 3′ relative to the promoter sequence that comprises a variable sequence that varies within the library; wherein a same cis element sequence is employed with a given reporter sequence within the library of constructs. According to this embodiment, the cells are optionally mammalian cells.

According to any of the construct, expression vector and cell library embodiments, the reporter sequences may comprise priming sequences 5′ and 3′ relative to the variable sequences. These priming sequences are optionally conserved within the library.

Also according to any of the construct, expression vector and cell library embodiments, the library may comprises at least 2, 3, 4, 5, 10, 20, 50, 100 or more different cis elements.

Also according to any of the construct, expression vector and cell library embodiments, the cis element sequence may comprise at least two, three, four or more copies of the cis element.

Also according to any of the construct, expression vector and cell library embodiments, an individual copy of the cis element may optionally have a length between about 5 and 100 base pairs, a length between about 5 and 75 base pairs, or a length between about 5 and 50 base pairs. An individual copy of the cis element may also have other lengths as described herein.

Also according to any of the construct, expression vector and cell library embodiments, the variable sequence of the reporter sequence may optionally be at least 15 bases in length, at least 25 bases in length, or at least 50 bases in length. The variable sequence of the reporter sequence may also optionally be between 15 and 2000 bases in length, between 25 and 2000 bases in length, between 50 and 2000 bases in length. Depending on the application, other lengths may also be used as described herein.

Also according to any of the construct, expression vector and cell library embodiments, it is noted that the different reporter sequences may optionally encode different reporter proteins.

Kits are also provided that comprise a construct, expression vector and/or cell library according to the present invention.

In one embodiment, the kit further comprises a library of hybridization probes for detecting by a hybridization assay a plurality of the variable sequences of the reporter sequences comprised in the library of nucleic acid constructs and/or complements of the variable sequences. Optionally, the library of hybridization probes may be immobilized in an array.

In another embodiment, the kit comprises primers for the priming sequences 5′ and 3′ relative to the variable sequences.

In yet another embodiment, the kit comprises a look-up table, in physical form and/or stored on computer readable media, the look-up table identifying a relationship between the reporter sequences in the library and the cis elements in the library and/or the transcription factors that bind to the cis elements in the library.

Methods for detecting transcription factors are also provided.

In one embodiment, a method is provided for identifying multiple different activated transcription factors in a cell sample, the method comprising: transducing or transfecting a cell sample to comprise a library of constructs, each construct comprising a cis element sequence comprising one or more copies of a cis element to which a transcription factor is capable of binding, the cis element sequence varying within the library of constructs, a promoter sequence 3′ relative to the cis element sequence, and a reporter sequence 3′ relative to the promoter sequence that comprises a variable sequence that varies within the library, wherein a same cis element sequence is employed with a given reporter sequence within the library of constructs; forming mRNA transcription products by those of the transduced or transfected cells in which an activated transcription factor is present that binds to the cis element of the construct present in the cell and activates transcription of the reporter sequence of the construct present in the cell; determining which reporter sequences are comprised within the mRNA transcription products; and determining which activated transcription factors are present in the cell sample based on which reporter sequences were transcribed.

In another embodiment, a method is provided for characterizing a cell type of a cell sample, the method comprising: identifying multiple different activated transcription factors in a cell sample by transducing or transfecting a cell sample to comprise a library of constructs, each construct comprising a cis element sequence comprising one or more copies of a cis element to which a transcription factor is capable of binding, the cis element sequence varying within the library of constructs, a promoter sequence 3′ relative to the cis element sequence, and a reporter sequence 3′ relative to the promoter sequence that comprises a variable sequence that varies within the library, wherein a same cis element sequence is employed with a given reporter sequence within the library of constructs, forming mRNA transcription products by those of the transduced or transfected cells in which an activated transcription factor is present that binds to the cis element of the construct present in the cell and activates transcription of the reporter sequence of the construct present in the cell, determining which reporter sequences are comprised within the mRNA transcription products, and determining which activated transcription factors are present in the cell sample based on which reporter sequences were transcribed; and using the combination of multiple different activated transcription factors identified as being present in a cell sample to identify the cell type of the cell sample.

According to this embodiment, using the identified combination of multiple different activated transcription factors may optionally comprise comparing the identified combination of multiple different activated transcription factors to combinations of different activated transcription factors known to be present in known cell types.

Also according to this embodiment, examples of known cell types include, but are not limited to diseased and/or healthy cells of a given cell type.

Also according to this embodiment, the combinations of different activated transcription factors present in known cell types may optionally be determined by transducing or transfecting a cell sample of a known cell type to comprise a library of constructs, each construct comprising a cis element sequence comprising one or more copies of a cis element to which a transcription factor is capable of binding, the cis element sequence varying within the library of constructs, a promoter sequence 3′ relative to the cis element sequence, and a reporter sequence 3′ relative to the promoter sequence that comprises a variable sequence that varies within the library, wherein a same cis element sequence is employed with a given reporter sequence within the library of constructs, forming mRNA transcription products by those of the transduced or transfected cells of the known cell type in which an activated transcription factor is present that binds to the cis element of the construct present in the cell and activates transcription of the reporter sequence of the construct present in the cell, determining which reporter sequences are comprised within the mRNA transcription products, and determining which activated transcription factors are present in the cell sample of the known cell type based on which reporter sequences were transcribed.

In another embodiment, a method is provided for diagnosing a disease state in a cell sample, the method comprising: identifying multiple different activated transcription factors in a cell sample by transducing or transfecting a cell sample to comprise a library of constructs, each construct comprising a cis element sequence comprising one or more copies of a cis element to which a transcription factor is capable of binding, the cis element sequence varying within the library of constructs, a promoter sequence 3′ relative to the cis element sequence, and a reporter sequence 3′ relative to the promoter sequence that comprises a variable sequence that varies within the library, wherein a same cis element sequence is employed with a given reporter sequence within the library of constructs, forming mRNA transcription products by those of the transduced or transfected cells in which an activated transcription factor is present that binds to the cis element of the construct present in the cell and activates transcription of the reporter sequence of the construct present in the cell, determining which reporter sequences are comprised within the mRNA transcription products, and determining which activated transcription factors are present in the cell sample based on which reporter sequences were transcribed; and comparing the combination of multiple different activated transcription factors identified as being present in a cell sample to combinations of multiple different activated transcription factors known to be present in diseased and healthy cell samples.

In another embodiment, a method is provided for screening for transcription factor modulators, the method comprising: taking a cell library comprising a library of constructs, each construct comprising a cis element sequence comprising one or more copies of a cis element to which a transcription factor is capable of binding, the cis element sequence varying within the library of constructs, a promoter sequence 3′ relative to the cis element sequence, and a reporter sequence 3′ relative to the promoter sequence that comprises a variable sequence that varies within the library of constructs, wherein a same cis element sequence is employed with a given reporter sequence within the library of constructs; exposing the cell library to one or more different agents; forming mRNA transcription products by those cells in the library in which an activated transcription factor is present that binds to the cis element of the construct present in the cell and activates transcription of the reporter sequence of the construct present in the cell; determining which reporter sequences are comprised within the mRNA transcription products for the cells exposed to the different agents; and determining changes in transcription factor activity in response to the cells being exposed to the one or more different agents based on which reporter sequences were transcribed.

According to any of the above methods, the library of cells optionally comprises at least 10, 20, 50, 100 or more different cis elements and at least 10, 20, 50, 100 or more different reporter sequences.

Also according to any of the above methods, the cis element sequence optionally comprises at least two, three, four or more copies of the cis element.

Also according to any of the above methods, an individual copy of the cis element may optionally have a length between about 5 and 100 base pairs, between about 5 and 75 base pairs, or between about 5 and 50 base pairs.

Also according to any of the above methods, the variable sequence of the reporter sequence may optionally be at least 15, 25, or 50 bases in length.

Also according to any of the above methods, the variable sequence of the reporter sequence may optionally be between 15 and 2000 bases in length, between 25 and 2000 bases in length or between 50 and 2000 bases in length.

Also according to any of the above methods, the cell samples may optionally comprise mammalian cells. The cells samples optionally are obtained from a human.

Also according to any of the above methods, determining which activated transcription factors are present in the cell sample may optionally be based on which reporter sequences were transcribed comprises using a look-up table to correlate transcribed reporter sequences with activated transcription factors.

Also according to any of the above methods, determining which of the reporter sequences were transcribed may optionally comprise reverse transcribing the mRNA transcription products to form cDNA and determining which of the reporter sequences or compliments thereof are comprised within the cDNA. According to this variation, the reporter sequences may comprise priming sequences 5′ and 3′ relative to the variable sequences, the method may further comprise amplifying the cDNA. Also according to this variation, determining which of the reporter sequences or compliments thereof are comprised within the cDNA may comprise sequencing the cDNA. Determining which of the reporter sequences or compliments thereof are comprised within the cDNA may also comprise performing a hybridization assay using a library of hybridization probes to detect the reporter sequences and/or compliments thereof. In this variation, the library of hybridization probes may optionally be immobilized in an array.

Also according to any of the above methods, the reporter sequences may optionally encode reporter proteins that the cells express from the mRNA transcription products. In such instances, determining which reporter sequences are comprised within the mRNA transcription products may optionally comprise determining which of the reporter proteins were expressed. Determining which of the reporter proteins were expressed may optionally comprise employing a library of antibodies capable of binding to the reporter proteins to detect the expressed reporter proteins. It is noted that the library of antibodies may optionally be immobilized in an array.

### BRIEF DESCRIPTION OF THE DRAWINGS

FIG. 1A provides a flow diagram for a method for identifying which of a plurality of activated transcription factors are present in a sample of cells based on detecting mRNA.

FIG. 1B provides a flow diagram for a method for identifying which of a plurality of activated transcription factors are present in a sample of cells based on detecting expressed reporter proteins.

FIG. 2 illustrates a look-up table for an exemplary library according to the present invention, the table providing a list of different transcription factors that can be detected by the library, the cis elements for the different transcription factors, and the reporter sequences associated with the different cis elements in the library.

FIG. 3 illustrates an array of hybridization probes attached to a solid support where different hybridization probes

### DETAILED DESCRIPTION OF THE INVENTION

The present invention relates to rapid and efficient methods for the parallel identification of multiple different activated transcription factors in a biological sample.

In one embodiment, a library of nucleic acid constructs is provided. Each construct comprises a cis element to which a transcription factor is capable of binding, a promoter 3′ relative to the cis element, and a reporter sequence 3′ relative to the promoter. The cis elements and reporter sequences each vary within the library of constructs. However, the cis elements and reporter sequences vary dependently with each other within the library of constructs in the sense that a same reporter sequence is present when a given cis element is present. This allows transcription and optionally translation of a given reporter sequence to be indicative of the presence of a particular transcription factor that bound to the cis element and activated transcription of the construct.

As will be described herein, the variable portion of the reporter sequence may itself be detected in order to detect the transcription of the reporter sequence. In such instances, the reporter sequence optionally comprises a primer at the 3′ end so that cDNA reverse transcribed from an mRNA transcription product of the construct may be amplified. The reporter sequence optionally also comprises a primer at the 5′ end also for use in amplifying the cDNA. One or more 3′ and 5′ primers may be used in the library. However, by using only one 3′ primer and one 5′ primer, all cDNA derived from expression of the construct library can be amplified together using just two priming sequences.

As will also be described herein, the variable portion of the reporter sequence may be used to encode a reporter protein that is to be detected. In such instances, the variable portion of the reporter sequence should be positioned in an open reading frame 3′ relative to the promoter so that the reporter protein may be expressed.

In another embodiment, a library of constructs according to the present invention are incorporated into a vector that is able to transduce or transfect a cell sample to form a library of cells capable of transcribing the reporter sequence as mRNA when a transcription factor binds to the cis element and induces expression.

In yet another embodiment, a library of constructs according to the present invention has been transduced or transfected into a cell sample to form a library of cells capable of transcribing the reporter sequence as mRNA when a transcription factor binds to the cis element and induces expression. Optionally, the cells also express reporter proteins encoded by the reporter sequences.

Methods are also provided for the identification of multiple different activated transcription factors in a biological sample using the libraries according to the present invention.

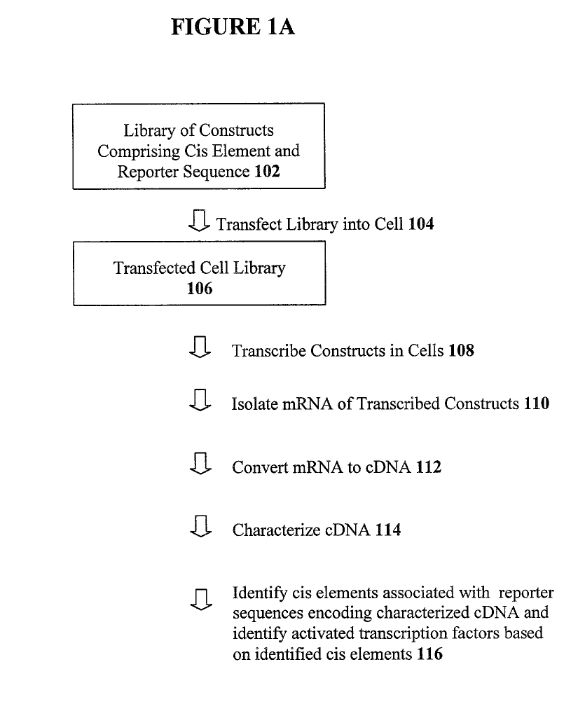

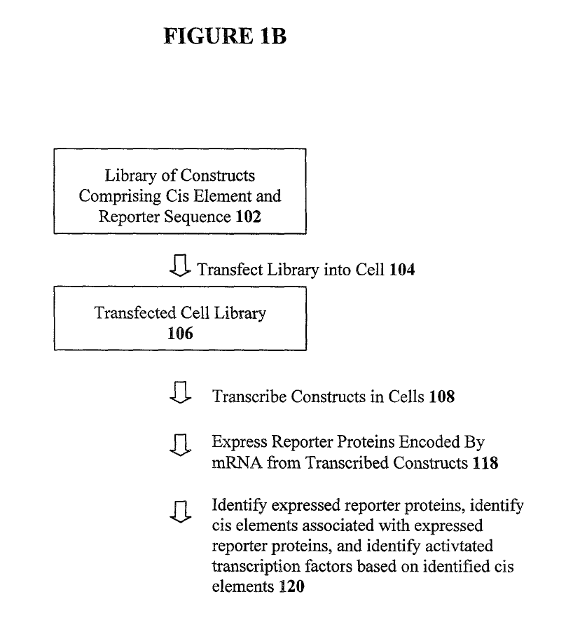

In embodiments of the method, illustrated in FIGS. 1A and 1B, a cell library 106 is provided that has been transduced or transfected 104 with a library of constructs 102, each construct comprising a cis element to which a transcription factor is capable of binding, a promoter 3′ relative to the cis element, and a reporter sequence 3′ relative to the promoter. The cis elements and reporter sequences each vary dependently with each other within the library of constructs. It is noted that the process of forming the library of constructs, as well as the process of forming a library of vectors and transducing or transfecting a cell sample with the library of vectors may also be part of the method.

mRNA transcription products encoded by the reporter sequences are produced by those cells in the library in which an activated transcription factor binds to the cis element of the construct and activates transcription of the reporter sequence 108.

In one variation, shown in FIG. 1A, mRNA from cells in the library is then isolated 110 and reverse transcribed to form cDNA 112. Sequences comprising at least the variable portion of the reporter sequences or compliments thereof that are comprised within the cDNA are then determined 114. As noted previously, the reporter sequences may optionally comprise 5′ and 3′ priming sequences that facilitate amplification of the cDNA to assist with their detection.

Knowing which of the reporter sequences are comprised within the cDNA allows one to determine which reporter sequences were transcribed. This allows one to determine which activated transcription factors were present in the cell library around the time that the mRNA was isolated from the cells in the library 116. This is because transcription of a given reporter sequence requires that an activated transcription factor bind to the cis element associated with that reporter sequence.

In another variation of the method, illustrated in FIG. 1B, the variable portion of the reporter sequence encodes a reporter protein. According to this variation, the mRNA are translated in the cells such that the reporter proteins encoded by the mRNA are expressed 118. In such instances, the reporter sequences encoding the reporter proteins should be positioned in an open reading frame 340 relative to the promoter so that the reporter proteins may be expressed.

The reporter proteins are then isolated and detected, most commonly by the use of antibodies that are selective for the expressed reporter proteins 120. By detecting the reporter proteins expressed from the mRNA, one is able to determine which mRNA were present and hence which reporter sequences were expressed. Since expression of a reporter protein requires that an active transcription factor bind to the cis element associated with the reporter protein, expression of a given reporter protein indicates that a corresponding activated transcription factor was present in the cell to bind to the cis factor and cause transcription of the construct encoding that reporter protein.

As will be described herein in greater detail, there are a great many applications that arise from being able to effectively monitor which activated transcription factors are present in a cell sample at a given time or under given conditions. With the assistance of the methods of the present invention, it is thus possible to rapidly and effectively monitor the presence of multiple different activated transcription factors in parallel.

The present invention also relates to various compositions, libraries, kits, and devices for use in conjunction with the various methods and applications of the present invention. Further aspects of the invention will be appreciated to those of ordinary skill in the art.

1. Libraries Comprising Cis Element—Reporter Sequence Constructs

Libraries of constructs are provided, each construct comprises a cis element to that a transcription factor is capable of binding, a promoter 3′ relative to the cis element, and a reporter sequence 3′ relative to the promoter. These libraries may be in the forms of a library of nucleic acid sequences, a library of vectors comprising the constructs (e.g., plasmid or phage), or a library of cells that have been transduced or transfected by the vector library to include the library of constructs.

The cis elements and reporter sequences each vary within the library of constructs. However, the cis elements and reporter sequences vary dependently with each other within the library of constructs. Namely, a same reporter sequence is paired with a given cis element. This allows transcription and optionally translation of a given reporter sequence to be indicative of the presence of a particular transcription factor that bound to the cis element and activated transcription of the construct.

Libraries of constructs can be assembled comprising a myriad of different cis element—reporter sequence pairings. The set of different cis element—reporter sequence pairings included in a given library will depend on the desired purpose of performing the method. In some instances, it will be desired to monitor transcription factor activity for a large number of transcription factors that may be present in the cell. In other instances, it may be desired to monitor the transcription factor activity of a selected, smaller group of transcription factors. In other instances, the number of cis element—reporter sequence pairings used in the library may be for all or some of the different transcription factors present in the cell in which the construct is introduced. A given library may comprise at least 2, 3, 4, 5, 10, 20, 50, 100, 250, or more different cis element—reporter sequence pairings. The upper limit on the number of different constructs that may be incorporated into a library is limited only by the number of cis element-activated transcription factor pairs that are known for a given cell type.

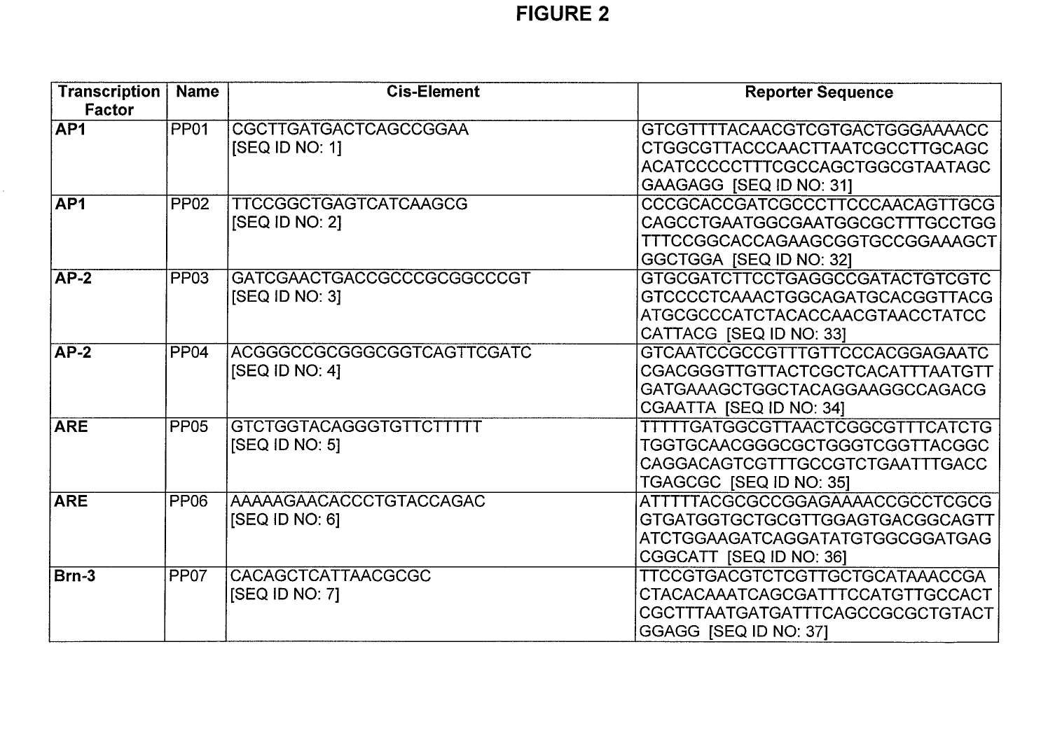

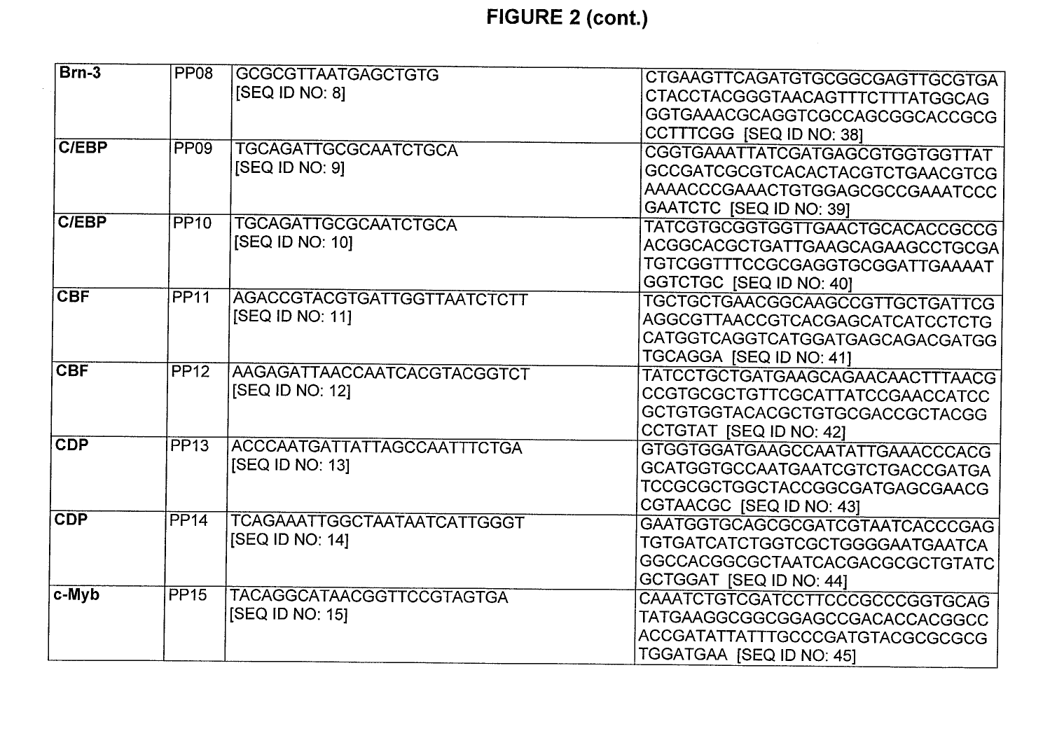

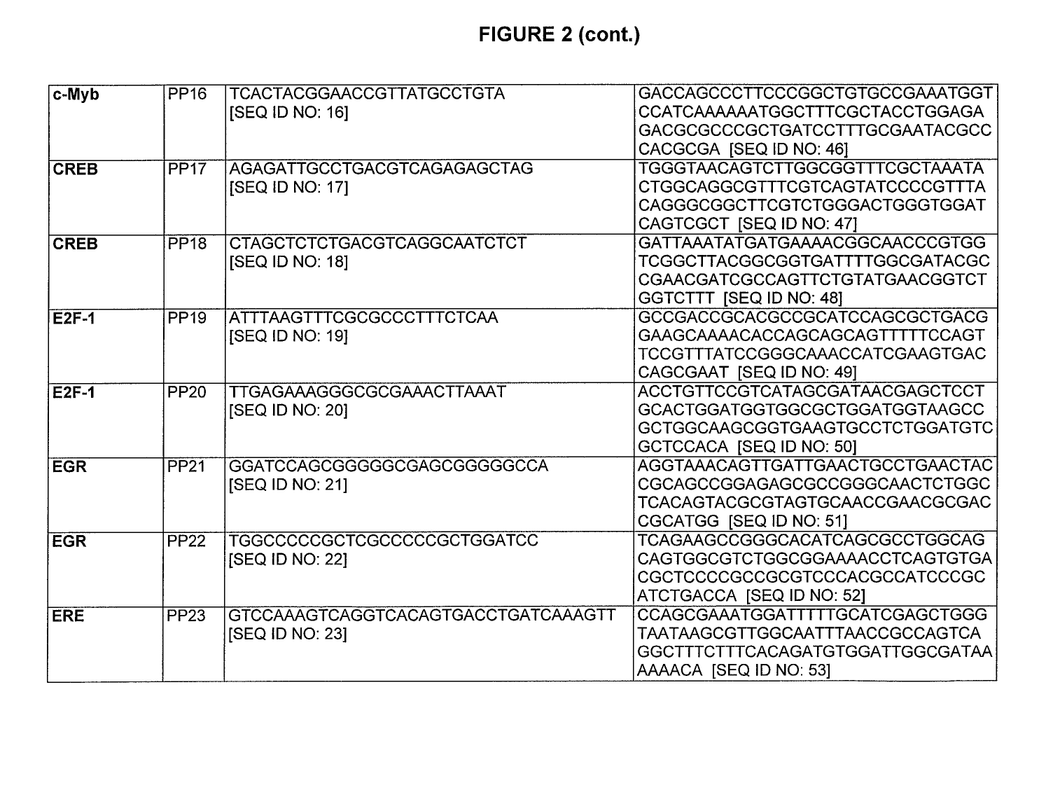

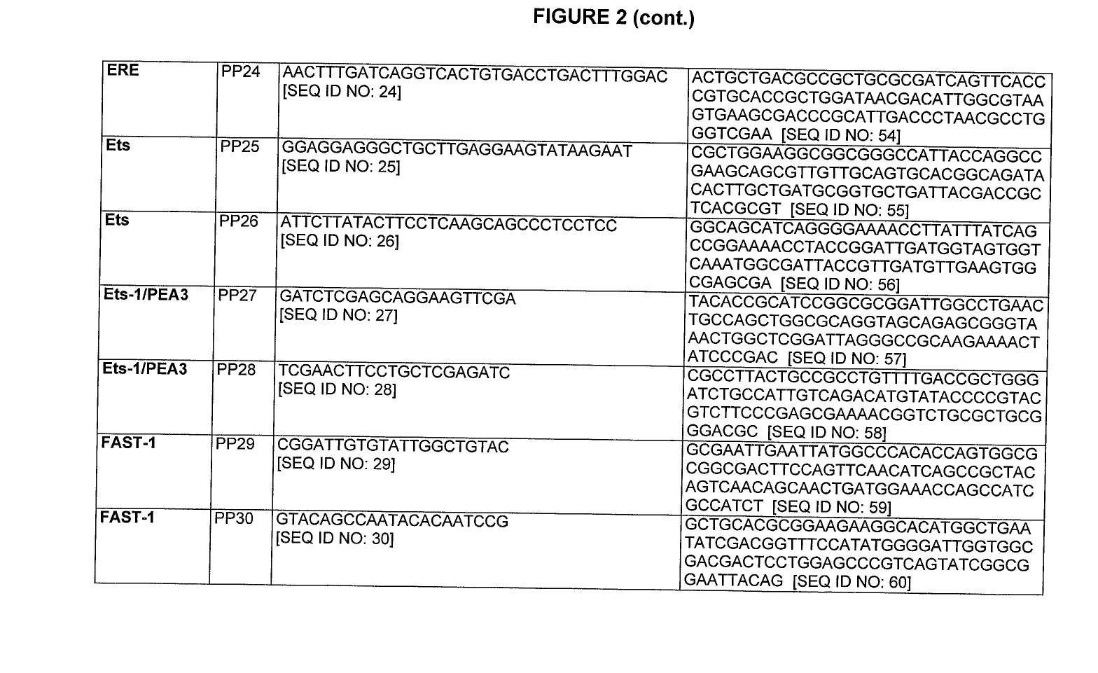

As illustrated in FIG. 2, different transcription factors are known that each bind to a different cis element. A different reporter sequence is assigned to each different cis element. Since the reporter sequences do not need to have any functional relationship with either the cis elements or their associated transcription factors, the reporter sequences in the library can be arbitrarily assigned to the different cis elements.

In this instance, the reporter sequences shown in FIG. 2 are 100 base pair fragments of the beta-galactosidase gene from E.coli. Longer or shorter fragments can also be employed as has already been indicated.

The E.coli beta-galactosidase gene is an attractive choice for a source of reporter sequences because it is known to have limited homology with human genes. It is noted that this approach can be used to expand the number of reporter sequences. For example, different genes from E. coli and genes from different organisms can be used.

As has been described, when a transcription factor binds to the cis element of a construct present in a cell, the reporter sequence downstream of the cis element is transcribed as mRNA.

As described in regard to FIG. 1A, the mRNA that is produced may be isolated and converted to cDNA. Reporter sequences comprised within the cDNA may be determined and used to identify which activated transcription factors are present in the cells. This is accomplished by using a look-up table, such as FIG. 2, that provides correlations between reporter sequences, transcription factors and cis elements.

As described in regard to FIG. 1B, the reporter sequences may encode reporter proteins that may be expressed from the mRNA and detected. The detected reporter proteins may be used to identify which activated transcription factors are present in the cells. This is accomplished by using a look-up table that identifies correlations between reporter proteins, transcription factors and cis elements.

Individual copies of the cis elements used in the constructs of the libraries preferably have a length between about 5 and 100 base pairs, more preferably between about 5 and 75 base pairs, more preferably between about 5 and 50 base pairs, more preferably between about 5 and 40 base pairs, and most preferably a length between about 5 and 35 base pairs. It is noted that the length of the cis elements may be otherwise varied from these ranges, as needed.

The optimal lengths for the individual copies of the cis elements may vary within the library depending on the particular transcription factor that binds to the cis element. Optionally, one may evaluate the optimal length for a given cis element for a given transcription factor. This may be performed, for example, using a traditional gel shift assay.

In order to facilitate binding of transcription factors to the cis elements, two, three, four or more copies of the cis element are preferably included in the constructs 5′ relative to the promoter.

Any promoter sequence that requires a cis element to activate transcription may be used in the constructs of the present invention. Examples of suitable promoters include, but are not limited to, thymidine kinase (TK), insulin promoter, human cytomegalovirus (CMV) promoter and its early promoter, simian virus SV40 promoter, Rous sarcoma virus LTR promoter, the chicken cytoplasmic β-actin promoter, promoters derived from immunoglobulin genes, bovine papilloma virus and adenovirus. A large number of other minimal promoters are known in the art and may also be used.

The reporter sequence is positioned 3′ relative to the promoter. Binding of transcription factors to the cis elements results in the reporter sequence being transcribed to produce mRNA. As discussed elsewhere, transcription of the reporter sequence is detected in order to evidence the presence of a transcription factor that can bind to the cis element associated with the reporter sequence that was transcribed.

In some instances, cDNA reverse transcribed from the mRNA is detected in order to detect the transcription of the reporter sequence. In such instances, it is advantageous for the reporter sequence to comprise 3′ and 5′ primers that allow the reporter sequence to be amplified relative to non-construct related cDNA that may also be present. This enhances the signal of the reporter sequences and diminishes the relative signal from false positive signals that the non-construct related cDNA could create.

Optionally, the reporter sequences positioned between the primers can be made to be different lengths. As a result, the cDNA may be amplified using the primers, and then detected based on their size, for example by using gel electrophoresis to perform the separation. Sequencing of the reporter sequences may also be performed, but is more tedious.

One or more 3′ and 5′ primers may be used in the library. However, by using only one 3′ primer and one 5′ primer, all cDNA derived from expression of the construct library can be amplified together using the two primers.

When the reporter sequence is to be detected via cDNA reverse transcribed from mRNA, the variable portions of the reporter sequences should be sufficiently long that the different reporter sequences employed in the library can be differentiated. Meanwhile, it is also desirable that the reporter sequences not be very long in order to avoid issues regarding transcribing, amplifying, and sequencing long sequences. For certain detection techniques, such as array detection, the reporter sequence is preferably not very long. In one embodiment, the reporter sequence is at least 15, 20, 25 35, or 50 bases in length. In another embodiment, the reporter sequence is less than 2000 bases, 1000 bases, 500 bases 250 bases or 100 bases in length.

In other instances, the mRNA encodes a reporter protein that is expressed by the cells. In such instances, the mRNA is detected by detecting a reporter protein that is encoded by the reporter sequence and hence the mRNA transcribed from the reporter sequence. In order for the reporter protein to be expressed, the reporter sequence should be positioned in an open reading frame 3′ relative to the promoter.

As noted, the libraries of constructs may be in the form of a library of vectors that may be used to transduce or transfect a cell sample with a construct library such that the cells are able to express the reporter sequences under the control of an associated cis element. Accordingly, the construct library may be incorporated into any vector that may be used to transduce or transfect cells in which transcription factor activity is to be detected.

The cis elements comprised in the construct library are preferably native relative to the cells used to form the cell library. Transcription factors and their associated cis elements are native to prokaryotes and eukaryotes, including fungi, plants, and animals, including mammals. Accordingly, this wide range of cells may be used to as the source of cell samples to form cell libraries transformed or transfected to include the construct library. Similarly, the vector library may comprise any vector that is able to transduce or transfect a cell sample to generate a library of cells that comprise the construct library.

The expression vector may be a mammalian express vector that can be used to express the construct library in mammalian cells. Examples of suitable mammalian cell lines include, but are not limited to, various COS cell lines, HeLa cells, myeloma cell lines, and CHO cell lines.

Typically, a mammalian expression vector includes certain expression control sequences, such as an origin of replication, a cis element, a promoter, as well as necessary processing signals, such as ribosome binding sites, RNA splice sites, polyadenylation sites, and transcriptional terminator sequences. The design of these vectors is well known in the art and can be readily adapted for the present invention.

The expression vectors containing the construct library can be transferred into the host cell by methods known in the art, depending on the type of host cells. Examples of transfection techniques include, but are not limited to, calcium phosphate transfection, calcium chloride transfection, lipofection, electroporation, and microinjection.

The construct library may also be inserted into a viral vector such as adenoviral vector that can replicate in various mammalian cells such as HeLa cells.

2. Method for Detecting Activated Transcription Factors

Methods are also provided for the identification of multiple different activated transcription factors in a cell sample.

According to embodiments of the method, a cell sample to be analyzed is transduced or transfected with a library of constructs according to the present invention.

mRNA transcription products encoded by the reporter sequences of the constructs are produced by those cells in the library in which an activated transcription factor binds to the cis element of the construct and activates transcription of the reporter sequence. The mRNA transcription products are then characterized, either by characterizing cDNA reversed transcribed from the mRNA or by expressing the mRNA. Both detection routes are described herein in greater detail.

As illustrated in regard to FIG. 2, knowing which sequences encoded by the reporter sequences are comprised within either the cDNA or reporter proteins allows one to determine which reporter sequences were transcribed. This allows one to determine which activated transcription factors were present in the cell library since transcription of a given reporter sequence requires that an activated transcription factor bind to the cis element associated with that reporter sequence.

A. Detection of Transcription Activators by Detection of cDNA

As noted, mRNA transcription products may be detected by detecting cDNA reverse transcribed from the mRNA transcription products. In order to facilitate analysis of the cDNA, it is desirable to amplify the cDNA. This can be accomplished by employing priming sequences 3′ and 5′ relative to the reporter sequence so that the reporter sequences can be readily amplified from within the cDNA.

The reporter sequences, preferably amplified, may be detected by a wide variety of methods. For example, the reporter sequences positioned between the primers may be made to have different lengths. This would allow the cDNA to be amplified using the primers, and then detected based on their size, for example by using gel electrophoresis to perform the separation. Sequencing of the reporter sequences may also be performed, but is more tedious. Alternatively, since the reporter sequences are known, they can be detected by hybridization to hybridization probes comprising the reporter sequences. Since the cDNA is duplexed, the complement to the reporter sequence may also be detected. According to this method, detection of a particular transcription factor is accomplished by detecting the formation of a duplex between the cDNA and a hybridization probe comprising at least a portion of the variable portion of the reporter sequence, or a complement thereof.

A wide variety of assays have been developed for performing hybridization assays and detecting the formation of duplexes that may be used in the present invention. For example, hybridization probes with a fluorescent dye and a quencher where the fluorescent dye is quenched when the probe is not hybridized to a target and is not quenched when hybridized to a target oligonucleotide may be used. Such fluorescer-quencher probes are described in, for example, U.S. Pat. No. 6,070,787 and S. Tyagi et al., “Molecular Beacons: Probes that Fluoresce upon Hybridization”, Dept. of Molecular Genetics, Public Health Research Institute, New York, N.Y., Aug. 25, 1995, each of which are incorporated herein by reference. By attaching different fluorescent dyes to different hybridization probes, it is possible to determine which reporter sequences from the library formed complexes based on which fluorescent dyes are present (e.g, fluorescent dye and quencher on hybridization probe). A difficulty arises however when using multiple different fluorescers in a single hybridization assay. Namely, there is a limited number of different fluorescers that may be spectrally resolved. As a result, a limited number of different reporter sequences can be detected at the same time, for example only as many as five to ten.

i. Hybridization Arrays for Detecting Reporter Sequences in cDNA

A desirable aspect of the present invention is its ability to detect a large number of transcription factors in parallel. In support of this, it is desirable to use detection approaches that support the detection of a large number of different sequences in parallel. One such approach involves the use of an array of hybridization probes immobilized on a solid support. The hybridization probes comprise sequences that are complementary to at least a portion of the reporter sequences (or their complements) and thus are able to hybridize to the different reporter sequences present in the construct library.

In order to enhance the sensitivity of the hybridization array, the immobilized probes preferably provide at least 2, 3, 4, 5 or more copies of at least a portion of the reporter sequences and/or their complements According to the present invention, the hybridization probes immobilized on the array preferably are at least 10, 15, 25, 30, 40 or 50 or more nucleotides in length. By immobilizing hybridization probes on a solid support that comprise one or more copies of a complement to at least a portion of the reporter sequences and/or their complements, the hybridization probes serve as immobilizing agents for the reporter sequences and/or their complements, each different hybridization probe being designed to selectively immobilize a different reporter sequence.

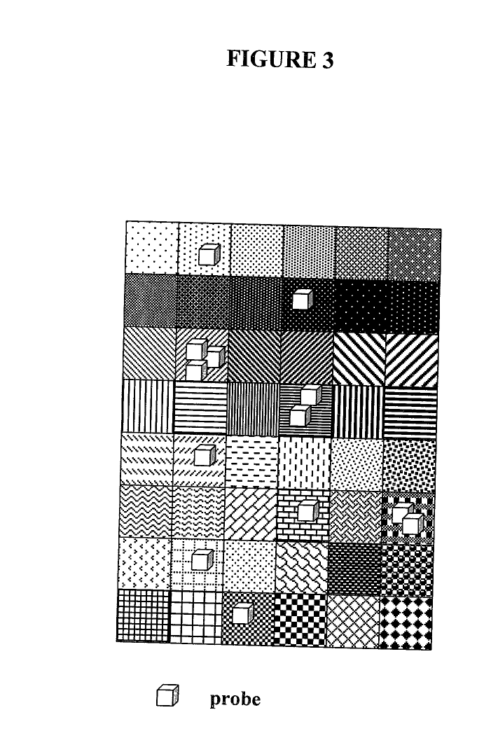

FIG. 3 illustrates an array of hybridization probes attached to a solid support where different hybridization probes are attached to discrete, different regions of the array. Each different region of the array comprises one or more copies of a same hybridization probe that incorporates a sequence that is complementary to a different reporter sequence or a complement of the reporter sequence. As a result, the hybridization probes in a given region of the array can selectively hybridize to and immobilize a different reporter sequence.

By detecting which regions the isolated transcription factor probes hybridize to on the array, one can determine which reporter sequences are present and hence which activated transcription factors were present in the sample.

The hybridization arrays can be designed and used to study transcription factor activation in a variety of biological processes, including cell proliferation, differentiation, transformation, apoptosis, drug treatment, and others described herein.

Numerous methods have been developed for attaching hybridization probes to solid supports in order to perform immobilized hybridization assays and detect target oligonucleotides in a sample. Numerous methods and devices are also known in the art for detecting the hybridization of a target oligonucleotide to a hybridization probe immobilized in a region of the array. Examples of such methods and device for forming arrays and detecting hybridization include, but are not limited to those described in U.S. Pat. Nos. 6,197,506, 6,045,996, 6,040,138, 5,424,186, 5,384,261, each of which are incorporated herein by reference.

Several modifications may be made to the hybridization arrays known in the art in order to customize the hybridization arrays for use in detecting activated transcription factors through the characterization of reporter sequences.

Since the hybridization probe arrays of the present invention are designed to hybridize to the reporter sequences in the library, the composition of the hybridization probes in the array should complement the reporter sequences and their complements that may be present in the cDNA. As discussed above, depending on the application, different numbers and combinations of reporter sequences may be included in a library and thus may be present in the cDNA.

A significant feature of the present invention is the ability to detect multiple different transcription factors at the same time. This ability arises from the number of different cis elements used in the library. A given array of hybridization probes preferably can be used to detect at least 2, 3, 4, 5, 10, 20, 30, 50, 100, 250 or more different reporter sequences. The upper limit on the number of different reporter sequences that the array of hybridization probes may detect is limited only by the number of cis elements and transcription factors to be detected.

a. Procedure for Performing Hybridization Using Array

Provided below is a description of a procedure that may be used to hybridize reporter sequences amplified from cDNA to a hybridization array. It is noted that the below procedure may be varied and modified without departing from other aspects of the invention.

An array membrane having hybridization probes attached for the reporter sequences is first placed into a hybridization bottle. The membrane is then wet by filling the bottle with deionized H₂O. After wetting the membrane, the water is decanted. Membranes that may be used as array membranes include any membrane to which a hybridization probe may be attached. Specific examples of membranes that may be used as array membranes include, but are not limited to NYTRAN membrane (Schleicher & Schuell), BIODYNE membrane (Pall), and NYLON membrane (Roche Molecular Biochemicals).

5 ml of prewarmed hybridization buffer is then added to each hybridization bottle containing an array membrane. The bottle is then placed in a hybridization oven at 42° C. for 2 hr. An example of a hybridization buffer that may be used is EXPHYP by CLONTECH.

After incubating the hybridization bottle, a thermal cycler may be used to denature the hybridization probes by heating the probes at 90° C. for 3 min, followed by immediately chilling the hybridization probes on ice.

The isolated reporter sequences are then added to the hybridization bottle. Hybridization is preferably performed at 42° C. overnight.

After hybridization, the hybridization mixture is decanted from the hybridization bottle. The membrane is then washed repeatedly.

In one embodiment, washing includes using 60 ml of a prewarmed first hybridization wash that preferably comprises 2×SSC/0.5% SDS. The membrane is incubated in the presence of the first hybridization wash at 42° C. for 20 min with shaking. The first hybridization wash solution is then decanted and the membrane washed a second time. A second hybridization wash, preferably comprising 0.1×SSC/0.5% SDS is then used to wash the membrane further. The membrane is incubated in the presence of the second hybridization wash at 42° C. for 20 min with shaking. The second hybridization wash solution is then decanted and the membrane washed a second time.

b. Procedure for Detecting Array Hybridization

The following describes a procedure that may be used to detect reporter sequences isolated on the hybridization array. It is noted that each membrane should be separately hybridized, washed and detected in separate containers in order to prevent cross contamination between samples. It is also noted that it is preferred that the membrane is not allowed to dry during detection.

According to the procedure, the membrane is carefully removed from the hybridization bottle and transferred to a new container containing 30 ml of 1× blocking buffer. The dimensions of each container is preferably about 4.5″×3.5″, equivalent in size to a 200 μL pipette-tip container. Table 1 provides an embodiment of a blocking buffer that may be used.

TABLE 1

1X Blocking Buffer:
Blocking reagent: 1%
0.1 M Maleic acid
0.15 M NaCl
Adjusted with NaOH to pH 7.5

It is noted that the array membrane may tend to curl adjacent its edges. It is desirable to keep the array membrane flush with the bottom of the container.

The array membrane is incubated at room temperature for 30 min with gentle shaking. 1 ml of blocking buffer is then transferred from each membrane container to a fresh 1.5 ml tube. 3 μl of Streptavidin-AP conjugate is then added to the 1.5 ml tube and is mixed well. The contents of the 1.5 ml tube is then returned to the container and the container is incubated at room temperature for 30 min.

The membrane is then washed three times at room temperature with 40 ml of 1× detection wash buffer, each 10 min. Table 2 provides an embodiment of a 1× detection wash buffer that may be used.

TABLE 2

1 X Detection wash buffer:
10 mM Tris-HCl, pH 8.0
150 mM NaCl
0.05% Tween-20

30 ml of 1× detection equilibrate buffer is then added to each membrane and the combination is incubated at room temperature for 5 min. Table 3 provides an embodiment of a 1× detection equilibrate buffer that may be used.

TABLE 3

1 X Detection equilibrate buffer:
0.1 M Tris-HCl pH 9.5
0.1 M NaCl

The resulting membrane is then transferred onto a transparency film. 3 ml of CPD-Star substrate, produced by Applera, Applied Biosystems Division, is then pipetted onto the membrane.

A second transparency film is then placed over the first transparency. It is important to ensure that substrate is evenly distributed over the membrane with no air bubbles. The sandwich of transparency films are then incubated at room temperature for 5 min.

The CPD-Star substrate is then shaken off and the films are wiped. The membrane is then exposed to Hyperfilm ECL, available from Amersham-Pharmercia. Alternatively, a chemiluminescence imaging system may be used such as the ones produced by ALPHA INNOTECH. It may be desirable to try different exposures of varying lengths of time (e.g., 2-10 min).

The hybridization array may be used to obtain a quantitative analysis of the number of reporter sequences present. For example, if a chemiluminescence imaging system is being used, the instructions that come with that system's software should be followed. If Hyperfilm ECL is used, it may be necessary to scan the film to obtain numerical data for comparison.

B. Detection of Transcription Activators by Detection of Reporter Proteins

As noted, the mRNA transcription products may also be detected by detecting reporter proteins encoded by the mRNA transcription products. In such instances, it is important for the constructs to be designed such that the encoded reporter proteins are expressed.

Once expressed, the reporter proteins may be detected by a wide variety of methods known in the art for detecting proteins. Most preferably, the reporter proteins are detected without having to isolate and purify the proteins. This may be accomplished by using proteins such as antibodies that are capable of selectively binding to the different reporter proteins that may be expressed in the library.

A variety of different techniques are known in the art for detecting protein-protein complexes. In one embodiment, the reporter proteins are detected using an immobilized array of antibodies or other proteins that can selectively bind to the different reporter proteins. FIG. 3, described above, illustrates an array of hybridization probes attached to a solid support where different hybridization probes are attached to discrete, different regions of the array. In this embodiment, antibodies for the different reporter proteins, instead of hybridization probes, may be attached to the discrete, different regions of the array. Preferably, the antibodies are immobilized at different positions on a solid support so that there is no cross interactions among them. As a result, the formation of an antibody-reporter sequence complex in a given region of the array indicates the presence of that reporter sequence.

Numerous methods have been developed for attaching antibodies to solid supports in order to perform immobilized protein binding assays and detect proteins in a sample, e.g., U.S. Pat. No. 6,197,599 which is incorporated herein by reference. For example, the antibodies may be immobilized on a solid support directly or indirectly. The antibodies may be directly deposited at high density on a support using similar technology as was developed for making high density DNA microarray, e.g., Shalon et al., Genome Research; 6(7): 639645 (1996). The antibodies can also be immobilized indirectly on the support, for example, by printing proteins that the antibodies can bind to onto a support. The antibodies are then immobilized on the support through their interactions with printed proteins. An advantage of this approach is that the constant regions of the antibodies can be made to bind to the printed protein. This leaves the variable regions of the antibodies (antigen-binding domains) fully exposed to interact with reporter proteins. Recombinant fusion proteins can also be immobilized through the interaction between their tags and the ligands printed on the support.

An important characteristic of protein arrays is that all agents are immobilized at predetermined positions, so that each agent can be identified by its position. After antibodies are immobilized, the support can be treated with 5% non-fat milk or 5% bovine serum albumin for several hours in order to block later non-specific protein binding.

A significant feature of the present invention is the ability to detect multiple different transcription factors at the same time. This ability arises from the number of different cis elements used in the library. A given array of antibodies preferably recognizes at least 2, 3, 5, 10, 20, 30, 50, 100, 250 or more different reporter proteins. The upper limit on the number of different reporter proteins that the array of antibodies may detect is limited only by the number of cis elements and transcription factors to be detected.

3. Applications for Detecting Activated Transcription Factors

With the assistance of the methods of the present invention, it is thus possible to rapidly and effectively monitor the presence of multiple different activated transcription factors in parallel. By better understanding which cells express which genes and how different conditions influence gene expression, fundamental questions of biology can be answered. Thus, by being able to rapidly and efficiently detect multiple activated transcription factors at the same time, the present invention avails itself to numerous valuable applications relating to the monitoring of gene expression. Some of these applications are described herein. Other applications will be apparent to those of ordinary skill.

a. Characterization of Cell Type

It is noted that different organisms will also express different activated transcription factors. Characterizing the mixture of different activated transcription factors expressed by a particular organism (e.g., a culture of bacteria) can be used to identify the particular organism. This application of the method of the present invention may be particularly useful for rapidly characterizing microbes such as bacteria and tissue with different disease states (e.g., types of malignancies).

By detecting and optionally quantifying which activated transcription factors are present in a cell sample, the methods of the present invention allow one to identify which genes are being expressed and to what extent each gene is being expressed. As a result, the present invention allows one to rapidly characterize a cell type based on which activated transcription factors are present and at what levels.

One embodiment of this application of the present invention thus relates to a method for characterizing a cell type by transducing or transfecting cells of an unknown cell type with a library of constructs according to the present invention and detecting which reporter sequences are transcribed as mRNA by detecting either cDNA derived from the mRNA or reporter proteins expressed from the mRNA. By identifying which transcription factors are present, one is able to obtain information about the unknown cell type.

b. Determining the Functions of Different Genes

Despite the fact that each cell in the human body contains the same set of genes, the human body is comprised of a wide diversity of different cell types that work in concert to form the human body. The wide diversity of cell types present in the human body and other multicellular organisms is due to variations between cells regarding which genes are expressed, the level at which the genes are expressed, and the conditions under which the genes are expressed. The present invention provides the unique ability of rapidly determining which of a great number of genes are expressed by numerous different cell types. By being able to determine which genes are expressed by which cell types, the functions of different genes can be deduced.

c. Diagnosis of Disease States

Certain disease states may be caused and/or characterizable by certain genes being expressed or not expressed as compared to normal cells. Other disease states may result from and/or be characterizable by certain genes being transcribed at different levels as compared to normal cells.

By being able to rapidly monitor the expression levels of multiple different genes, the present invention provides an accurate method for diagnosing certain disease states known to be associated with the expression non-expression, reduced expression, and/or elevated expression of one or more genes. Conversely, by comparing the expression non-expression, reduced expression, and/or elevated expression of one or more genes in normal and abnormal cells, present invention facilitates the association of one or more genes with certain disease states. By understanding that a particular disease state is caused by a different expression (higher or lower) of one or more proteins, it should be possible to remedy the disease state by increasing or decreasing the expression of the one or more proteins, by administering the one or more proteins or, if particular proteins are overexpressed, by inhibiting the one or more proteins.

One embodiment of this application of the present invention thus relates to a method for diagnosing a disease state, the method comprising transducing or transfecting cells to be diagnosed with a library of constructs according to the present invention and detecting which reporter sequences are transcribed as mRNA by detecting either cDNA derived from the mRNA or reporter proteins expressed from the mRNA. By identifying which transcription factors are present, one is able to obtain information about the disease state of the cells.

d. Compound Screening for Drug Candidates

Being able to monitor transcription factor activity for multiple different transcription factors at the same time is of great importance to developing a better understanding of different roles that various transcription factors play. In addition, monitoring multiple different transcription factors at the same time allows one to rapidly screen for compounds that influence transcription factor activity, referred to herein as a “transcription factor modulator.”

The present invention may thus be used as a high throughput screening assay for transcription factor modulators that either up- or down-regulate genes by influencing the synthesis and activation of transcription factors for those genes.

One embodiment of this application of the present invention thus relates to a method for monitoring an affect different agents have on transcription factor activity, the method comprising: taking a library of cells comprising a construct library according to the present invention; exposing cells from the library to different agents; and determining which transcription factors are present in the cells exposed to the different agents by detecting which reporter sequences are transcribed by the different cells. The method may further comprise the use of controls, i.e., the identification of which transcription factors are present when the cells are not exposed to an agent. The method may also further comprise taking one or more agents and identifying an affect dosage has on transcription factor activity for the one or more agents.

By having a further understanding of what agents modulate transcription factor activity, such agents may be more effectively used for in vitro modification of signal transduction, transcription, splicing, and the like, e.g., as tools for recombinant methods, cell culture modulators, etc. More importantly, such agents can be used as lead compounds for drug development for a variety of conditions, including as antibacterial, antifungal, antiviral, antineoplastic, inflammation modulatory, or immune system modulatory agents. Accordingly, being able to monitor transcription factor activity for multiple different factors has great use for screening agents to identify lead compounds for pharmaceutical or other applications.

Indeed, because gene expression is fundamental in all biological processes, including cell division, growth, replication, differentiation, repair, infection of cells, etc., the ability to monitor transcription factor activity and identify agents that modulate their activity can be used to identify drug leads for a variety of conditions, including neoplasia, inflammation, allergic hypersensitivity, metabolic disease, genetic disease, viral infection, bacterial infection, fungal infection, or the like. In addition, compounds that specifically target transcription factors in undesired organisms such as viruses, fungi, agricultural pests, or the like, can serve as fungicides, bactericides, herbicides, insecticides, and the like. Thus, the range of conditions that are related to transcription factor activity includes conditions in humans and other animals, and in plants, e.g., for agricultural applications.

As used herein, the term “transcription factor modulator” refers to any molecule or complex of more than one molecule that affects the regulatory region. The present invention contemplates screens for synthetic small molecule agents, chemical compounds, chemical complexes, and salts thereof as well as screens for natural products, such as plant extracts or materials obtained from fermentation broths. Other molecules that can be identified using, the screens of the invention include proteins and peptide fragments, peptides, nucleic acids and oligonucleotides (particularly triple-helix-forming oligonucleotides), carbohydrates, phospholipids and other lipid derivatives, steroids and steroid derivatives, prostaglandins and related arachadonic acid derivatives, etc.

Existing methods for monitoring gene expression typically monitor down-stream expression processes by measuring mRNA or the resulting gene product. However, why a particular mRNA or protein is expressed at higher or lower levels is not revealed by these methods. This is because a given compound can influence the formation of a transcription factor, influence the activation of the transcription factor, interact with the activated transcription factor, interact with the regulatory element to which the transcription factor binds, or interact with the mRNA that is produced.

By contrast, because the present invention is specific to detecting activated transcription factors, the present invention can be effectively used to screen for drugs that have a mechanism of action directly related to the expression and/or activation of transcription factors.

It should be noted that methods exist for measuring a transcription factor in a sample. However, because such methods detect the protein itself, they are unable to determine whether the transcription factor is activated, i.e., it is capable of binding to a regulatory element. By being able to detect whether multiple different transcription factors are activated, the present invention, when used in combination with an assay for detecting the amount of activated and unactivated transcription factor, allows one to evaluate specifically how a given compound influences the activation of different transcription factors.

The present invention may be used to screen large chemical libraries for modulator activity for multiple different transcription factors. For example, by exposing cells to different members of the chemical libraries, and performing the methods of the present invention, one is able to screen the different members of the library relative to multiple different transcription factors at the same time.

It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening involves testing a combinatorial library containing a large number of potential modulator compounds. A combinatorial chemical library may be a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Such combinatorial libraries are then screened to identify those library members (particular chemical species or subclasses) that modulate one or more transcription factors. The compounds thus identified can serve as conventional “lead compounds” or can themselves be used as potential or actual therapeutics for the one or more transcription factors whose activities the compounds modulate.

Preparation and screening of combinatorial libraries is well known to those of skill in the art. Such combinatorial libraries include, but are not limited to, peptide libraries (e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (PCT Publication No. WO 91/19735), encoded peptides (PCT Publication WO 93/20242), random bio-oligomers (PCT Publication No. WO 92/00091), benzodiazepines (U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with .beta.-D-glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see, Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. Nos. 5,506,337; benzodiazepines, 5,288,514, and the like).

Devices for the preparation of combinatorial libraries are also commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

Control reactions may be performed in combination with the libraries. Such optional control reactions are appropriate and increase the reliability of the screening. Accordingly, in a preferred embodiment, the methods of the invention include such a control reaction. The control reaction may be a negative control reaction that measures the transcription factor activity independent of a transcription modulator. The control reaction may also be a positive control reaction that measures transcription factor activity in view of a known transcription modulator.

By being able to screen multiple different transcription factors at the same time, not only is it possible to screen a large number of potential transcription modulators per day, it is also possible to screen any potential transcription modulator relative to a large number of different transcription factors. The ability to screen multiple different transcription factors at the same time thus greatly enhances the high throughput capabilities of this screening assay.

d. Evaluation of Drug Efficacy

Given that certain disease states may be caused by an unusual level of transcription of one or more genes, drugs may be designed to either stimulate or inhibit transcription in order make gene expression of diseased cells approach the gene expression of normal cells. A rapid and effective method for monitoring gene expression is thus highly advantageous for evaluating the effectiveness of a drug's ability to alter the transcription of one or more genes. The effectiveness of a drug being delivered to a site of action as well as the drug's efficacy in vivo can thus be evaluated with the assistance of the methods of the present invention.

Also of great concern when developing new drugs is the side effects that the drugs might have. One approach for screening drug candidates for undesirable side effects would be to employ the present invention to monitor how gene expression is altered in response to the administration of a drug candidate. By understanding how a candidate affects gene expression, candidates likely to have undesirable side affects can be rapidly identified.

Because the biological importance of transcription factors, they are ideal drug targets. Traditional transcription factor screening assays only detect one transcription factor at a time. As a result, existing assays are tremendously in efficient for detecting how a drug effects different gene expression. However, with the assistance of the present invention, it is now possible to screen hundreds and even thousands of transcription factors in a short amount of time in order to monitor how a given drug affects the expression of wide range of genes. The present invention will thus dramatically facilitate the screening process of identifying new drugs, characterizing their mechanism of the action, and screening for adverse side effects based on the drug's impact on expression.

4. Kits

A wide variety of kits may be designed for use with the present invention. In general, the kits of the present invention may comprise any combination of two or more libraries, devices, and/or reagents that may be used in combination to perform a method according to the present invention.

For example, in one embodiment, a kit is provided that comprises a library of constructs where the reporter sequence has 5′ and 3′ priming sequences, and primers for the 5′ and 3′ priming sequences that may be used to amplify the reporter sequences. It is noted that the library of constructs may be a nucleic acid, vector or cell library.

In another embodiment, a kit is provided that comprises a library of constructs and an array comprising immobilized hybridization probes for detecting all or a portion of the reporter sequences in the library and/or the complements. Again, it is noted that the library of constructs may be a nucleic acid, vector or cell library. Also, the kit may further comprise primers for amplifying the reporter sequences.

Other kits, beyond those exemplified herein can be readily envisioned by one of ordinary skill in the art, all of which are intended to fall within the scope of the present invention.

In general, it will be apparent to those skilled in the art that various modifications and variations can be made to the compositions, libraries, kits, and methods of the present invention without departing from the spirit or scope of the invention. Thus, it is intended that the present invention cover the modifications and variations of this invention provided they come within the scope of the appended claims and their equivalents.

What is claimed is:

1. A library of nucleic acid constructs, each construct comprising:
a cis element sequence comprising one or more copies of a cis element to which a transcription factor is capable of binding, the cis element sequence varying within the library of constructs;
a promoter sequence 3′ relative to the cis element sequence; and
a reporter sequence 3′ relative to the promoter sequence that comprises a variable sequence that varies within the library;
wherein a same cis element sequence is employed with a given reporter sequence within the library of constructs.

2. A library according to claim 1 wherein the reporter sequences comprise priming sequences 5′ and 3′ relative to the variable sequences.

3. A library according to claim 2 wherein the 5′ and 3′ priming sequences are conserved within the library.

4. A library according to claim 1 wherein the library comprises at least 10 different cis elements.

5. A library according to claim 1 wherein the library comprises at least 20 different cis elements.

6. A library according to claim 1 wherein the library comprises at least 50 different cis elements.

7. A library according to claim 1 wherein the library comprises at least 100 different cis elements.

8. A library according to claim 1 wherein the cis element sequence comprises at least two copies of the cis element.

9. A library according to claim 1 wherein the cis element sequence comprises at least three copies of the cis element.

10. A library according to claim 1 wherein the cis element sequence comprises at least four copies of the cis element.

11. A library according to claim 1 wherein an individual copy of the cis element has a length between about 5 and 100 base pairs.

12. A library according to claim 1 wherein an individual copy of the cis element has a length between about 5 and 75 base pairs.

13. A library according to claim 1 wherein an individual copy of the cis element has a length between about 5 and 50 base pairs.

14. A library according to claim 1 wherein the variable sequence of the reporter sequence is at least 15 bases in length.

15. A library according to claim 1 wherein the variable sequence of the reporter sequence is at least 25 bases in length.

16. A library according to claim 1 wherein the variable sequence of the reporter sequence is at least 50 bases in length.

17. A library according to claim 1 wherein the variable sequence of the reporter sequence is between 15 and 2000 bases in length.

18. A library according to claim 1 wherein the variable sequence of the reporter sequence is between 25 and 2000 bases in length.

19. A library according to claim 1 wherein the variable sequence of the reporter sequence is between 50 and 2000 bases in length.

20. A library according to claim 1 wherein the different reporter sequences encode different reporter proteins.

21. A library according to claim 20 wherein the reporter sequence is in an open reading frame relative to the promoter sequence.

22. A library according to claim 21 wherein the reporter sequence comprises a stop codon 3′ relative to sequence encoding reporter protein.

23. A library of expression vectors comprising:
a library of constructs, each construct comprising
a cis element sequence comprising one or more copies of a cis element to which a transcription factor is capable of binding, the cis element sequence varying within the library of constructs;
a promoter sequence 3′ relative to the cis element sequence; and
a reporter sequence 3′ relative to the promoter sequence that comprises a variable sequence that varies within the library of constructs;
wherein a same cis element sequence is employed with a given reporter sequence within the library of constructs.

24. A library according to claim 23 wherein the expression vectors are mammalian expression vectors.

25. A library according to claim 23 wherein the reporter sequences comprise priming sequences 5′ and 3′ relative to the variable sequences.

26. A library according to claim 23 wherein the library of constructs comprises at least 10 different cis elements.

27. A library according to claim 23 wherein the cis element sequence comprises at least two copies of the cis element.

28. A library according to claim 23 wherein the cis element sequence comprises at least three copies of the cis element.

29. A library according to claim 23 wherein the cis element sequence comprises at least four copies of the cis element.

30. A library according to claim 23 wherein an individual copy of the cis element has a length between about 5 and 100 base pairs.

31. A library according to claim 23 wherein the variable sequence of the reporter sequence is at least 15 bases in length.

32. A library according to claim 23 wherein the variable sequence of the reporter sequence is between 15 and 2000 bases in length.

33. A library according to claim 23 wherein the different reporter sequences encode different reporter proteins.

34. A library according to claim 33 wherein the reporter sequence is in an open reading frame relative to the promoter sequence.

35. A library according to claim 34 wherein the reporter sequence comprises a stop codon 3′ relative to sequence encoding reporter protein.

36. A library of cells transduced or transfected with a library of constructs, each construct comprising:
a cis element sequence comprising one or more copies of a cis element to which a transcription factor is capable of binding, the cis element sequence varying within the library of constructs;
a promoter sequence 3′ relative to the cis element sequence; and
a reporter sequence 3′ relative to the promoter sequence that comprises a variable sequence that varies within the library;
wherein a same cis element sequence is employed with a given reporter sequence within the library of constructs.

37. A library according to claim 36 wherein the cells are mammalian cells.

38. A library according to claim 36 wherein the reporter sequences comprise priming sequences 5′ and 3′ relative to the variable sequences.

39. A library according to claim 36 wherein the library of constructs comprises at least 10 different cis elements.

40. A library according to claim 36 wherein the cis element sequence comprises at least two copies of the cis element.

41. A library according to claim 36 wherein the cis element sequence comprises at least three copies of the cis element.

42. A library according to claim 36 wherein the cis element sequence comprises at least four copies of the cis element.

43. A library according to claim 36 wherein an individual copy of the cis element has a length between about 5 and 100 base pairs.

44. A library according to claim 36 wherein the variable sequence of the reporter sequence is at least 15 bases in length.

45. A library according to claim 36 wherein the variable sequence of the reporter sequence is between 15 and 2000 bases in length.

46. A library according to claim 36 wherein the different reporter sequences encode different reporter proteins.

47. A library according to claim 46 wherein the reporter sequence is in an open reading frame relative to the promoter sequence.

48. A library according to claim 47 wherein the reporter sequence comprises a stop codon 3′ relative to sequence encoding reporter protein.

49. A kit comprising
a library of nucleic acid constructs, each construct comprising:
a cis element sequence comprising one or more copies of a cis element to which a transcription factor is capable of binding, the cis element sequence varying within the library of constructs,
a promoter sequence 3′ relative to the cis element sequence, and
a reporter sequence 3′ relative to the promoter sequence that comprises a variable sequence that varies within the library,
wherein a same cis element sequence is employed with a given reporter sequence within the library of constructs; and
a library of hybridization probes for detecting by a hybridization assay a plurality of the variable sequences of the reporter sequences comprised in the library of nucleic acid constructs and/or complements of the variable sequences.

50. A kit according to claim 49, wherein the library of hybridization probes are immobilized in an array.

51. A kit according to claim 49 wherein the reporter sequences comprise priming sequences 5′ and 3′ relative to the variable sequences and the kit further comprises primers for the priming sequences.

52. A kit according to claim 49 wherein the library comprises at least 10 different reporter sequences.

53. A kit according to claim 52 wherein the library of hybridization probes comprises hybridization probes for detecting at least 10 different reporter sequences.

54. A kit according to claim 49 wherein the library of constructs comprises at least 20 different reporter sequences.

55. A kit according to claim 54 wherein the library of hybridization probes comprises hybridization probes for detecting at least 20 different reporter sequences.

56. A kit according to claim 49 wherein the library of constructs comprises at least 50 different reporter sequences.

57. A kit according to claim 54 wherein the library of hybridization probes comprises hybridization probes for detecting at least 50 different reporter sequences.

58. A kit comprising:
a library of expression vectors comprising
a library of nucleic acid constructs, each construct comprising:
a cis element sequence comprising one or more copies of a cis element to which a transcription factor is capable of binding, the cis element sequence varying within the library of constructs,
a promoter sequence 3′ relative to the cis element sequence, and
a reporter sequence 3′ relative to the promoter sequence that comprises a variable sequence that varies within the library,
wherein a same cis element sequence is employed with a given reporter sequence within the library of constructs; and
a library of hybridization probes for detecting by a hybridization assay a plurality of the variable sequences of the reporter sequences comprised in the library of nucleic acid constructs, and/or complements of the variable sequences.

59. A kit according to claim 58, wherein the library of expression vectors are mammalian expression vectors.

60. A kit according to claim 58, wherein the library of hybridization probes are immobilized in an array.

61. A kit according to claim 58 wherein the reporter sequences comprise priming sequences 5′ and 3′ relative to the variable sequences and the kit further comprises primers for the priming sequences.

62. A kit according to claim 58 wherein the library of expression vectors comprises at least 10 different reporter sequences.

63. A kit according to claim 62 wherein the library of hybridization probes comprises hybridization probes for detecting at least 10 different reporter sequences.

64. A kit according to claim 58 wherein the library of expression vectors comprises at least 20 different reporter sequences.

65. A kit according to claim 64 wherein the library of hybridization probes comprises hybridization probes for detecting at least 20 different reporter sequences.

66. A kit according to claim 58 wherein the library of expression vectors comprises at least 50 different reporter sequences.

67. A kit according to claim 64 wherein the library of hybridization probes comprises hybridization probes for detecting at least 50 different reporter sequences.

68. A kit comprising
a library of cells transduced or transfected with a library of constructs, each construct comprising:
a cis element sequence comprising one or more copies of a cis element to which a transcription factor is capable of binding, the cis element sequence varying within the library of constructs,
a promoter sequence 3′ relative to the cis element sequence, and
a reporter sequence 3′ relative to the promoter sequence that comprises a variable sequence that varies within the library,
wherein a same cis element sequence is employed with a given reporter sequence within the library of constructs; and
a library of hybridization probes for detecting by a hybridization assay a plurality of the variable sequences of the reporter sequences comprised in the library of nucleic acid constructs, and/or complements of the variable sequences.

69. A kit according to claim 68, wherein the library of cells are mammalian cells.

70. A kit according to claim 68, wherein the library of hybridization probes are immobilized in an array.

71. A kit according to claim 68 wherein the reporter sequences comprise priming sequences 5′ and 3′ relative to the variable sequences and the kit further comprises primers for the priming sequences.

72. A kit according to claim 68 wherein the library of cells comprises at least 10 different reporter sequences.

73. A kit according to claim 72 wherein the library of hybridization probes comprises hybridization probes for detecting at least 10 different reporter sequences.

74. A kit according to claim 68 wherein the library of cells comprises at least 20 different reporter sequences.

75. A kit according to claim 74 wherein the library of hybridization probes comprises hybridization probes for detecting at least 20 different reporter sequences.

76. A kit according to claim 68-wherein the library of cells comprises at least 50 different reporter sequences.

77. A kit according to claim 74 wherein the library of hybridization probes comprises hybridization probes for detecting at least 50 different reporter sequences.

78. A kit comprising
a library of nucleic acid constructs, each construct comprising:
a cis element sequence comprising one or more copies of a cis element to which a transcription factor is capable of binding, the cis element sequence varying within the library of constructs,
a promoter sequence 3′ relative to the cis element sequence, and
a reporter sequence 3′ relative to the promoter sequence that comprises a variable sequence that varies within the library,
wherein a same cis element sequence is employed with a given reporter sequence within the library of constructs; and
a look-up table, in physical form and/or stored on computer readable media, the look-up table identifying a relationship between the reporter sequences in the library and the cis elements in the library and/or the transcription factors that bind to the cis elements in the library.

79. A kit comprising:
a library of expression vectors comprising
a library of nucleic acid constructs, each construct comprising:
a cis element sequence comprising one or more copies of a cis element to which a transcription factor is capable of binding, the cis element sequence varying within the library of constructs,
a promoter sequence 3′ relative to the cis element sequence, and
a reporter sequence 3′ relative to the promoter sequence that comprises a variable sequence that varies within the library,
wherein a same cis element sequence is employed with a given reporter sequence within the library of constructs; and
a look-up table, in physical form and/or stored on computer readable media, the look-up table identifying a relationship between the reporter sequences in the library of constructs and the cis elements in the library of constructs and/or the transcription factors in the library of constructs that bind to the cis elements.

80. A kit comprising
a library of cells transduced or transfected with a library of constructs, each construct comprising:
a cis element sequence comprising one or more copies of a cis element to which a transcription factor is capable of binding, the cis element sequence varying within the library of constructs,
a promoter sequence 3′ relative to the cis element sequence, and
a reporter sequence 3′ relative to the promoter sequence that comprises a variable sequence that varies within the library,
wherein a same cis element sequence is employed with a given reporter sequence within the library of constructs; and
a look-up table, in physical form and/or stored on computer readable media, the look-up table identifying a relationship between the reporter sequences in the library of constructs and the cis elements in the library of constructs and/or the transcription factors in the library of constructs that bind to the cis elements.


SEQUENCE LISTING (60 sequences)

SEQ ID NO: 1 - DNA, 21 bp, Artificial Sequence
  PPO1 Cis-Element
cgcttgatgactcagccggaa

SEQ ID NO: 2 - DNA, 21 bp, Artificial Sequence
  PP02 Cis-Element
ttccggctgagtcatcaagcg

SEQ ID NO: 3 - DNA, 26 bp, Artificial Sequence
  PP03 Cis-Element
gatcgaactgaccgcccgcggcccgt

SEQ ID NO: 4 - DNA, 26 bp, Artificial Sequence
  PP04 Cis-Element
acgggccgcgggcggtcagttcgatc

SEQ ID NO: 5 - DNA, 23 bp, Artificial Sequence
  PP05 Cis-Element
gtctggtacagggtgttcttttt

SEQ ID NO: 6 - DNA, 23 bp, Artificial Sequence
  PP06 Cis-Element
aaaaagaacaccctgtaccagac

SEQ ID NO: 7 - DNA, 18 bp, Artificial Sequence
  PPO7 Cis-Element
cacagctcattaacgcgc

SEQ ID NO: 8 - DNA, 18 bp, Artificial Sequence
  PP08 Cis-Element
gcgcgttaatgagctgtg

SEQ ID NO: 9 - DNA, 20 bp, Artificial Sequence
  PP09 Cis-Element
tgcagattgcgcaatctgca

SEQ ID NO: 10 - DNA, 20 bp, Artificial Sequence
  PP10 Cis-Element
tgcagattgcgcaatctgca

SEQ ID NO: 11 - DNA, 27 bp, Artificial Sequence
  PP11 Cis-Element
agaccgtacgtgattggttaatctctt

SEQ ID NO: 12 - DNA, 27 bp, Artificial Sequence
  PP12 Cis-Element
aagagattaaccaatcacgtacggtct

SEQ ID NO: 13 - DNA, 27 bp, Artificial Sequence
  PP13 Cis-Element
acccaatgattattagccaatttctga

SEQ ID NO: 14 - DNA, 27 bp, Artificial Sequence
  PP14 Cis-Element
tcagaaattggctaataatcattgggt

SEQ ID NO: 15 - DNA, 25 bp, Artificial Sequence
  PP15 Cis-Element
tacaggcataacggttccgtagtga

SEQ ID NO: 16 - DNA, 25 bp, Artificial Sequence
  PP16 Cis-Element
tcactacggaaccgttatgcctgta

SEQ ID NO: 17 - DNA, 27 bp, Artificial Sequence
  PP17 Cis-Element
agagattgcctgacgtcagagagctag

SEQ ID NO: 18 - DNA, 27 bp, Artificial Sequence
  PP18 Cis-Element
ctagctctctgacgtcaggcaatctct

SEQ ID NO: 19 - DNA, 25 bp, Artificial Sequence
  PP19 Cis-Element
atttaagtttcgcgccctttctcaa

SEQ ID NO: 20 - DNA, 25 bp, Artificial Sequence
  PP20 Cis-Element
ttgagaaagggcgcgaaacttaaat

SEQ ID NO: 21 - DNA, 27 bp, Artificial Sequence
  PP21 Cis-Element
ggatccagcgggggcgagcgggggcca

SEQ ID NO: 22 - DNA, 27 bp, Artificial Sequence
  PP22 Cis-Element
tggcccccgctcgcccccgctggatcc

SEQ ID NO: 23 - DNA, 35 bp, Artificial Sequence
  PP23 Cis-Element
gtccaaagtcaggtcacagtgacctgatcaaagtt

SEQ ID NO: 24 - DNA, 35 bp, Artificial Sequence
  PP24 Cis-Element
aactttgatcaggtcactgtgacctgactttggac

SEQ ID NO: 25 - DNA, 31 bp, Artificial Sequence
  PP25 Cis-Element
ggaggagggctgcttgaggaagtataagaat

SEQ ID NO: 26 - DNA, 31 bp, Artificial Sequence
  PP26 Cis-Element
attcttatacttcctcaagcagccctcctcc

SEQ ID NO: 27 - DNA, 21 bp, Artificial Sequence
  PP27 Cis-Element
gatctcgagcaggaagttcga

SEQ ID NO: 28 - DNA, 21 bp, Artificial Sequence
  PP28 Cis-Element
tcgaacttcctgctcgagatc

SEQ ID NO: 29 - DNA, 21 bp, Artificial Sequence
  PP29 Cis-Element
cggattgtgtattggctgtac

SEQ ID NO: 30 - DNA, 21 bp, Artificial Sequence
  PP30 Cis-Element
gtacagccaatacacaatccg

SEQ ID NO: 31 - DNA, 100 bp, Artificial Sequence
  PP01 Reporter Sequence
gtcgttttacaacgtcgtgactgggaaaaccctggcgttacccaacttaatcgccttgcagcacatccccctttcgccagctggcgtaatagcgaagagg

SEQ ID NO: 32 - DNA, 100 bp, Artificial Sequence
  PP02 Reporter Sequence
cccgcaccgatcgcccttcccaacagttgcgcagcctgaatggcgaatggcgctttgcctggtttccggcaccagaagcggtgccggaaagctggctgga

SEQ ID NO: 33 - DNA, 100 bp, Artificial Sequence
  PP03 Reporter Sequence
gtgcgatcttcctgaggccgatactgtcgtcgtcccctcaaactggcagatgcacggttacgatgcgcccatctacaccaacgtaacctatcccattacg

SEQ ID NO: 34 - DNA, 100 bp, Artificial Sequence
  PP04 Reporter Sequence
gtcaatccgccgtttgttcccacggagaatccgacgggttgttactcgctcacatttaatgttgatgaaagctggctacaggaaggccagacgcgaatta

SEQ ID NO: 35 - DNA, 100 bp, Artificial Sequence
  PP05 Reporter Sequence
tttttgatggcgttaactcggcgtttcatctgtggtgcaacgggcgctgggtcggttacggccaggacagtcgtttgccgtctgaatttgacctgagcgc

SEQ ID NO: 36 - DNA, 100 bp, Artificial Sequence
  PP06 Reporter Sequence
atttttacgcgccggagaaaaccgcctcgcggtgatggtgctgcgttggagtgacggcagttatctggaagatcaggatatgtggcggatgagcggcatt

SEQ ID NO: 37 - DNA, 100 bp, Artificial Sequence
  PP07 Reporter Sequence
ttccgtgacgtctcgttgctgcataaaccgactacacaaatcagcgatttccatgttgccactcgctttaatgatgatttcagccgcgctgtactggagg

SEQ ID NO: 38 - DNA, 100 bp, Artificial Sequence
  PP08 Reporter Sequence
ctgaagttcagatgtgcggcgagttgcgtgactacctacgggtaacagtttctttatggcagggtgaaacgcaggtcgccagcggcaccgcgcctttcgg

SEQ ID NO: 39 - DNA, 100 bp, Artificial Sequence
  PP09 Reporter Sequence
cggtgaaattatcgatgagcgtggtggttatgccgatcgcgtcacactacgtctgaacgtcgaaaacccgaaactgtggagcgccgaaatcccgaatctc

SEQ ID NO: 40 - DNA, 100 bp, Artificial Sequence
  PP10 Reporter Sequence
tatcgtgcggtggttgaactgcacaccgccgacggcacgctgattgaagcagaagcctgcgatgtcggtttccgcgaggtgcggattgaaaatggtctgc

SEQ ID NO: 41 - DNA, 100 bp, Artificial Sequence
  PP11 Reporter Sequence
tgctgctgaacggcaagccgttgctgattcgaggcgttaaccgtcacgagcatcatcctctgcatggtcaggtcatggatgagcagacgatggtgcagga

SEQ ID NO: 42 - DNA, 100 bp, Artificial Sequence
  PP12 Reporter Sequence
tatcctgctgatgaagcagaacaactttaacgccgtgcgctgttcgcattatccgaaccatccgctgtggtacacgctgtgcgaccgctacggcctgtat

SEQ ID NO: 43 - DNA, 100 bp, Artificial Sequence
  PP13 Reporter Sequence
gtggtggatgaagccaatattgaaacccacggcatggtgccaatgaatcgtctgaccgatgatccgcgctggctaccggcgatgagcgaacgcgtaacgc

SEQ ID NO: 44 - DNA, 100 bp, Artificial Sequence
  PP14 Reporter Sequence
gaatggtgcagcgcgatcgtaatcacccgagtgtgatcatctggtcgctggggaatgaatcaggccacggcgctaatcacgacgcgctgtatcgctggat

SEQ ID NO: 45 - DNA, 100 bp, Artificial Sequence
  PP15 Reporter Sequence
caaatctgtcgatccttcccgcccggtgcagtatgaaggcggcggagccgacaccacggccaccgatattatttgcccgatgtacgcgcgcgtggatgaa

SEQ ID NO: 46 - DNA, 100 bp, Artificial Sequence
  PP16 Reporter Sequence
gaccagcccttcccggctgtgccgaaatggtccatcaaaaaatggctttcgctacctggagagacgcgcccgctgatcctttgcgaatacgcccacgcga

SEQ ID NO: 47 - DNA, 100 bp, Artificial Sequence
  PP17 Reporter Sequence
tgggtaacagtcttggcggtttcgctaaatactggcaggcgtttcgtcagtatccccgtttacagggcggcttcgtctgggactgggtggatcagtcgct

SEQ ID NO: 48 - DNA, 100 bp, Artificial Sequence
  PP18 Reporter Sequence
gattaaatatgatgaaaacggcaacccgtggtcggcttacggcggtgattttggcgatacgccgaacgatcgccagttctgtatgaacggtctggtcttt

SEQ ID NO: 49 - DNA, 100 bp, Artificial Sequence
  PP19 Reporter Sequence
gccgaccgcacgccgcatccagcgctgacggaagcaaaacaccagcagcagtttttccagttccgtttatccgggcaaaccatcgaagtgaccagcgaat

SEQ ID NO: 50 - DNA, 100 bp, Artificial Sequence
  PP20 Reporter Sequence
acctgttccgtcatagcgataacgagctcctgcactggatggtggcgctggatggtaagccgctggcaagcggtgaagtgcctctggatgtcgctccaca

SEQ ID NO: 51 - DNA, 100 bp, Artificial Sequence
  PP21 Reporter Sequence
aggtaaacagttgattgaactgcctgaactaccgcagccggagagcgccgggcaactctggctcacagtacgcgtagtgcaaccgaacgcgaccgcatgg

SEQ ID NO: 52 - DNA, 100 bp, Artificial Sequence
  PP22 Reporter Sequence
tcagaagccgggcacatcagcgcctggcagcagtggcgtctggcggaaaacctcagtgtgacgctccccgccgcgtcccacgccatcccgcatctgacca

SEQ ID NO: 53 - DNA, 100 bp, Artificial Sequence
  PP23 Reporter Sequence
ccagcgaaatggatttttgcatcgagctgggtaataagcgttggcaatttaaccgccagtcaggctttctttcacagatgtggattggcgataaaaaaca

SEQ ID NO: 54 - DNA, 100 bp, Artificial Sequence
  PP24 Reporter Sequence
actgctgacgccgctgcgcgatcagttcacccgtgcaccgctggataacgacattggcgtaagtgaagcgacccgcattgaccctaacgcctgggtcgaa

SEQ ID NO: 55 - DNA, 100 bp, Artificial Sequence
  PP25 Reporter Sequence
cgctggaaggcggcgggccattaccaggccgaagcagcgttgttgcagtgcacggcagatacacttgctgatgcggtgctgattacgaccgctcacgcgt

SEQ ID NO: 56 - DNA, 100 bp, Artificial Sequence
  PP26 Reporter Sequence
ggcagcatcaggggaaaaccttatttatcagccggaaaacctaccggattgatggtagtggtcaaatggcgattaccgttgatgttgaagtggcgagcga

SEQ ID NO: 57 - DNA, 100 bp, Artificial Sequence
  PP27 Reporter Sequence
tacaccgcatccggcgcggattggcctgaactgccagctggcgcaggtagcagagcgggtaaactggctcggattagggccgcaagaaaactatcccgac

SEQ ID NO: 58 - DNA, 100 bp, Artificial Sequence
  PP28 Reporter Sequence
cgccttactgccgcctgttttgaccgctgggatctgccattgtcagacatgtataccccgtacgtcttcccgagcgaaaacggtctgcgctgcgggacgc

SEQ ID NO: 59 - DNA, 100 bp, Artificial Sequence
  PP29 Reporter Sequence
gcgaattgaattatggcccacaccagtggcgcggcgacttccagttcaacatcagccgctacagtcaacagcaactgatggaaaccagccatcgccatct

SEQ ID NO: 60 - DNA, 100 bp, Artificial Sequence
  PP30 Reporter Sequence
gctgcacgcggaagaaggcacatggctgaatatcgacggtttccatatggggattggtggcgacgactcctggagcccgtcagtatcggcggaattacag